(19) United States
(12) Patent Application Publication
(10) Pub. No.: US 2005/0147736 A1
(43) Pub. Date: Jul. 7, 2005

(54) Using polypyrrole as the contrast pH detector to fabricate a whole solid-state pH sensing device

(75) Inventors: Shen-Kan Hsiung, Jungli City (TW); Jung-Chuan Chou, Douliou City (TW); Tai-Ping Sun, Jungli City (TW); Chung-We Pan, Paotso Village (TW)

Correspondence Address: APEX JURIS, PLLC, 13194 EDGEWATER LANE NORTHEAST, SEATTLE, WA, 98125, US

(73) Assignee: Chung Yuan Christian University, Jungli City (TW)

(21) Appl. No.: 10/750,072

(22) Filed: Dec. 31, 2003

(51) Int. Cl. A61L 2/00; B05D 5/12

(52) U.S. Cl. 427/58; 205/414; 427/126.1

(57) ABSTRACT

A process for fabricating a whole solid-state pH sensing device by using the polypyrrole as the contrast pH detector and a whole solid-state pH sensing device fabricated by the process are disclosed, wherein said device is a differential pair framework potential electrochemical sensing device fabricated by using a non-insulating solid-state inorganic ion-sensing membrane and a polypyrrole sensing membrane. The largest difference between the device of the present invention and the conventional potentiometric type pH sensor is that the sensor of the invention is a solid-state planar sensor. The differential pair framework uses tin dioxide as the ion-sensing membrane and the reference electrode, and uses a polypyrrole sensor as the differential sensor, wherein the sensitivity of tin dioxide is good and has a value up to 57 mV/pH, and the sensitivity of polypyrrole is about 27 mV/pH. These lead the sensitivity of the whole solid-state pH Sensing device to a value of 30 mV/pH and exhibits good linearity, so that the sensing device framework has practicability. Since the sensitivity of the polypyrrole can be controlled by means of its polymerization, a sensing device with controllable sensitivity can be fabricated for applying to the fabrication of a pH sensor or a biosensor.

### BACKGROUND OF THE INVENTION

1. Field of the Invention

The invention relates to a process for fabricating a whole solid-state pH sensing device using polypyrrole as the contrast pH detector and in particular, to an pH sensing device with lower sensitivity fabricated by using the polypyrrole, wherein the features of the polypyrrole can be varied by controlling its polymerization environment and hence sensing devices with various features can be fabricated, such that, when it is used to fabricate the whole solid-state pH sensing device, controlling of the feature of the whole solid-state pH sensing device can be realized.

2. Description of the Prior Art

Since there are many drawbacks on the practical application of the conventional organic quantitative analysis [1], e.g., complex operation, long analysis time, expensive equipments, inapplicable for the detection of a continuous process, etc., studies to find out a solution that can overcome disadvantages associated with the conventional quantitative analysis had been carried out. As a result, the biosensor is designed by combining the theories of biochemistry, electrical circuit, material science, optics, etc, to be a biosensor meeting requirements of various fields. The prototype of the biosensor provided by Clark et al, 1962[2] was a new detection analytical method of organic substance based on the theory of the specificity of enzyme to its substrate. Thereafter, Updike and Hicks in 1967 made a glucose sensor by immobilizing a glucose oxidase to form a membrane [3] and combining with a dissolved-oxygen electrode. Henceforth, an upsurge of biosensor study was evoked, including: Clark-type oxygen electrode, hydrogen peroxide electrode, hydrogen electrode, hydrogen ion electrode, ion selective electrode, ammonium ion electrode, carbon dioxide electrode, and ion sensitive field effect transistor (ISFET).

The ISFET is a semiconductor pH sensor whose primary principle is consisted of removing the metal on tgate of the metal-oxide semi-field effect transistor (MOSFET) and placing into an aqueous solution to allow the silicon dioxide layer that is exposed through removing the gate metal to contact with the aqueous solution, so as to detect the Zeta potential produced from the aqueous solution against the silicon dioxide layer such that the purpose of sensing the ion concentration in the aqueous solution can be achieved. The related studies on ISFET, such as the improvement of materials [4-6], the study and miniaturization of reference electrodes [7-9], the improvement of structures [10-11], and the like, had be discussed successively. Since the come out of the ISFET element, other applications are developed extensively, for example, detection of the pH value, ions such as potassium, sodium, calcium, chloride, fluoride and iodide ions, and the like in the blood [12-17], which are still mainly utilizing the primary principle of ISFET.

An extended gate field effect transistor (EGFET) is an element developed from ISFET, provided firstly by J. Spiegel [18], and unlike ISFET, the EGFET preserves the original gate in the MOSFET and has a sensing membrane plated on the other end extended from the -metal gate. Compared with ISFET, the EGFET has following advantages: (1) the electrostatic protection provided by the conductive wire onto the element; (2) elimination of the direct contact of the transistor of the element with the aqueous solution; and (3) the effect of light on the element being reduced.

A reference electrode is a type of electrochemical sensing device, which is an electrode used to establish a standard reference potential corresponding to the different standard potential of the solution to be detected. Its working principle is to utilize the feature that its surface potential remains stable in different solution and avoids the deviation of the sensitivity of the sensing device caused by different solutions detected. A reference electrode commonly used on an ordinary electrochemical sensing device is a calomel electrode or a silver/silver chloride electrode, but most those reference electrodes are wet reference electrodes, and therefore, those reference electrodes cannot take place the miniaturization, and must immerse into an associated buffer solution for a long period, which is inconvenient both for its use and storage. Hence, in order to achieve the objects of the miniaturized fabrication and dry storage, in recent years, the design of a reference electrode is an important study subject and there are related articles having discussions on this aspect. Referring to articles on pH ISFET, it is found that the miniaturization of a reference electrode is a present tendency of the sensing device development, while current ways of fabrication include: micro-electromechanical processing, silver/silver chloride membrane deposition, differential pair circuit design, and the like [19-22].

As patent regarding conventional techniques, there can be mentioned as following:

(1) Byung Ki Sohn, U.S. Pat. No. 5,309,085; Date of patent: May 3, 1994“Measuring circuit with a biosensor utilizing ion sensitive field effect transistors,” provided a read-out circuit for the ISFET biosensor. The circuit had advantages of being a simple structure and easy to integration. The circuit comprised two ISFET as inputs, one was an enzyme field effect transistor (enzyme EFT), and the other was the reference FET. The enzyme FET was constructed by immobilizing enzyme on the sensing gate of the ISFET. This circuit had various amplification functions to amplify the sensed output of the sensing device. The voltage variation of ISFET was raised through using an unsteady semi-reference electrode that could be affected by the change of the temperature so that the working characteristic of the device could be adjusted by changing the gain of read-out circuit. The ISFET biosensor could be provided on a single chip in combination with a measuring circuit to achieve the miniaturization of the sensing device.

(2) Teruaki Katsube, Shuichiro Yamaguchi, Naoto Uchida, Takeshi Shimomura, U.S. Pat. No. 5,296,122; Date of patent: Mar. 22, 1994“Apparatus for forming thin film,” provided a hydrophobic membrane to be used as the reference electrode of an ISFET. The hydrophobic membrane was grown on a substrate through a neutral plasma or by sputtering using the target of the hydrophobic membrane. The instrument equipments included: a vacuum chamber, an atom beam generator, a target base, a shield for growth controlling, and the like. The membrane was suitable for the use of the ion sensor, such as the ISFET and the enzyme sensor.

(3) Barry W. Benton, U.S. Pat. No. 5,833,824; Date of patent Nov. 10, 1998“Dorsal substrate guarded ISFET sensor”, provided an ISFET sensor for sensing the activity of ions in the solution. The sensor comprised a substrate and a semiconductor chip of the ISFET. The front surface of plate contacted with the solution and its rear surface faced to the surface of the substrate. There was a hole connecting the front surface and the rear face of the substrate. In the gate region of the ISFET, there was an ion-sensing region that contacted with the rear face, and brought the gate region contact with the solution via the hole.

(4) James G. Connery, Jr. Shaffer, W. Earl, U.S. Pat. No. 4,879,517; Date of patent: Nov. 7, 1989“Temperature compensation for potentiometrically operated ISFETS,” provided a temperature compensating circuit of the ISFET. The ISFET has fixed source voltage, drain voltage and drain current. Based on the effect of the Nernst temperature effect on the output of the ISFET and the neutral point of the sensing probe, the working condition of the sensing device was corrected to zero temperature potential so as to lower the effect of temperature on the sensing device, and fabricated a set of an ISFET and an FET to eliminate the deviation from the device fabrication.

(5) Hendrik H. v. d. Vlekkert, Nicolaas F. de Rooy, U.S. Pat. No. 4,691,167; Date of patent: Sep. 1, 1987“Apparatus for determining the activity of an ion (plon) in a liquid,” provided an apparatus for measuring the activity of ions in a solution. The device comprised a measuring circuit including an ISFET, a reference electrode, a temperature sensor and an amplifier that included an ISFET, a temperature sensor, and a control/calculation/memory circuit, and was able to set VGS, VDS, IDS parameters on constant values. The detection of the ion activity could be obtained by controlling those three parameters. Since the ion-sensitivity possessed a temperature variation feature, and there existed a function relationship between IDS and temperature, the circuit could use the function stored in the memory to control VGS to achieve the compensation of the temperature feature.

(6) Mathias Krauss, Beate Hildebrandt, Christian Kunath, Eberhard Kurth, U.S. Pat. No. 5,602,467; Date of patent Feb. 11, 1997“Circuit for measuring ion concentrations in solutions,” provided a framework for measuring the ion concentration in the solution by using an ISFET circuit layout. The circuit layout could expose the gate voltage difference of the FET and the parameter/environmental deviation caused by operation factors. The circuit layout comprised two measurement/test amplifiers, two ISFETs, and two identical FETs. The ISFET was connected to FET, and output from the first amplifier displayed the gate voltage change between two ISFETs and FET, and the second amplifier displayed the output difference of two ISFET. The output of the first amplifier was the ground reference electrode that connected to 4 reference electrodes. Thus the framework was capable of detecting the ion concentration.

According with related studies, it was found that both the -solid-state dry reference electrode and the planar sensing device framework are related problems needed to be solved presently. According with the framework of the invention, the dry storage of the sensing device and the planar framework can be achieved.

Accordingly, it can be seen that the above-described conventional techniques still have many drawbacks, and are not designed well, and need to be improved urgently.

In view of disadvantages derived from the above-described conventional sensing device, the present inventor had devoted to improve and innovate, and, after studying intensively for many years, developed successfully a process for fabricating a whole solid-state pH sensing device by using polypyrrole as the contrast pH detector according to the invention.

### SUMMARY OF THE INVENTION

The object of the invention is to provide a process for fabricating a whole solid-state pH-sensing device by using polypyrrole as the contrast pH detector, which sensing device is a planar ion sensor. The senor is fabricated by combining the semiconductor process and the polymerization of polypyrrole. The invention process fabricates a pH sensor with a lower sensitivity by using polypyrrole. The feature of the polypyrrole can be adjusted by controlling its polymerization environment and hence can fabricate a sensing device with various features ∘ Therefore, when applying to the fabrication of the whole solid-state pH sensing device, control of the feature of the whole solid-state pH sensing device can be realized. As the sensing electrode and reference electrode are fabricated by tin dioxide, both are semiconductor membrane material, so a solid-state planar framework can be produced. As the result, the sensor of the invention exhibits various advantages, such as solid-state device, planar framework, dry storage, easy fabrication, and the like.

The process for fabricating a whole solid-state pH sensing device by using polypyrrole as the contrast pH detector that can achieve the above-described objects comprises of depositing a solid-state sensing membrane on the substrate by means of a semiconductor coating technology, and polymerizing and fixing polypyrrole on the conductive solid-state membrane by means of an electrochemical polymerization technology. The process according to the invention comprises following steps:
Step 1: providing a clean washed the indium tin oxide glass;
Step 2: depositing a tin dioxide membrane by a sputtering machine
Step 3: touting the device
Step 4: sealing an appropriate sensing area by using a epoxy resin;
Step 5: then immersing the device into an electro-polymerization solution, and electro-polymerizing polypyrrole, and thus accomplishing the fabrication of the whole solid-state pH sensing device.

### BRIEF DESCRIPTION OF THE DRAWINGS

The drawings disclose an illustrative embodiment of the invention which serves to exemplify the various advantages and objects hereof, and are as follows:

FIG. 1(a) is the flow chart of the process for fabricating a whole solid-state pH sensing device by using polypyrrole as the contrast pH detector according to the invention;

FIG. 1(b) is the flow chart of the process for fabricating said polypyrrole sensor

FIG. 2(a) is the top view of a whole solid-state pH sensing device fabricated by using polypyrrole as the contrast pH detector;

FIG. 2(b) is the sectional view of a whole solid-state pH sensing device fabricated by using polypyrrole as the contrast pH detector;

FIG. 3 is a schematic view showing the measurement of electro-polymerizing potential of the polypyrrole;

FIG. 4 is a schematic view showing the measurement system of oxidizing potential of a conductive polypyrrole polymer;

FIG. 5 is a framework diagram showing the electro-polymerization system of polypyrrole on the pH sensing device;

FIG. 6(a) is the characteristic measuring framework diagram of the pH sensing device;

FIG. 6(b) is the characteristic measuring framework diagram of the differential pair framework sensing device;

FIG. 7 is a diagram showing the sensitivity calibration curve of the tin dioxide/indium tin oxide glass sensing device;

FIG. 8 is a diagram showing the sensitivity calibration curve of the polypyrrole/tin dioxide/indium tin oxide glass sensing device;

FIG. 9 is a diagram showing output signals of a whole solid-state pH sensing device fabricated by using polypyrrole as the contrast pH detector in different pH solutions; and

FIG. 10 is a diagram showing the sensitivity curve of a whole solid-state pH sensing device fabricated by using polypyrrole as the contrast pH detector.

### DETAILED DESCRIPTION OF THE PREFERRED EMBODIMENT

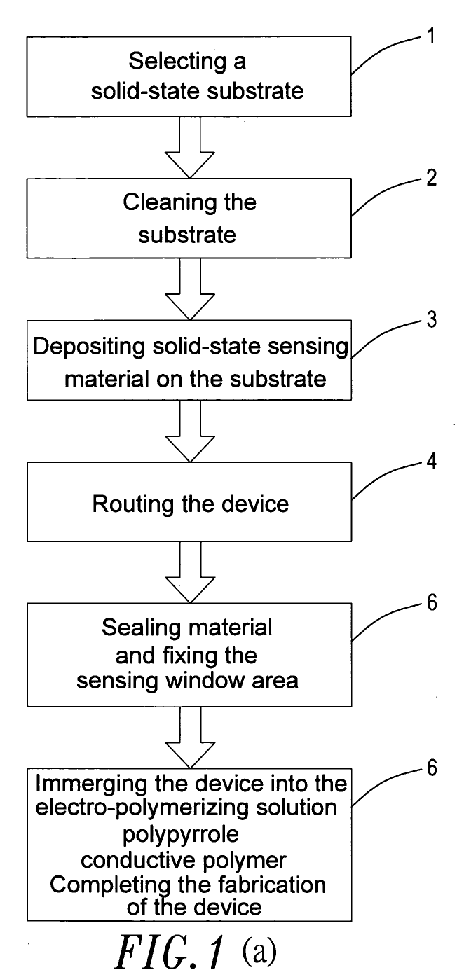

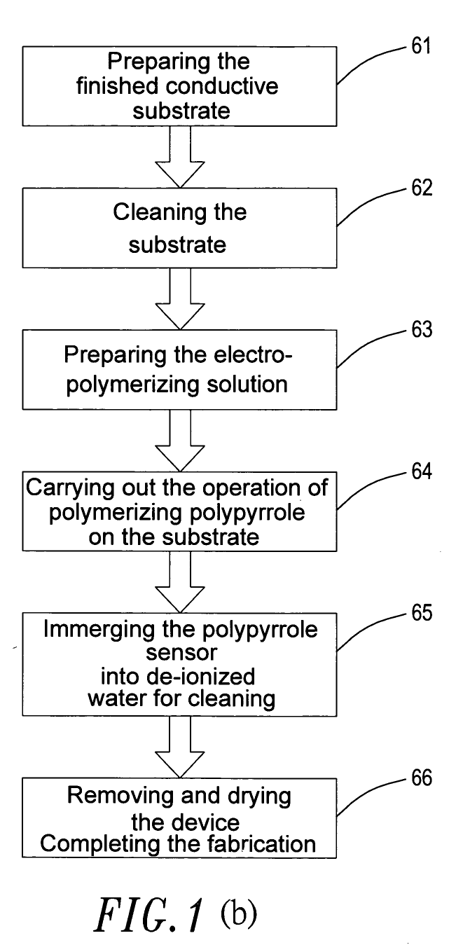

Referring to FIG. 1(a) and FIG. 1(b), there show the flow chart of the process for fabricating a whole solid-state pH sensing device by using polypyrrole as the contrast pH detector and the flow chart of the process for fabricating the polypyrrole sensor according to the invention, respectively. From the charts it can be seen that the process for fabricating a whole solid-state pH sensing device by using polypyrrole as the contrast pH detector according to the invention comprises of depositing a solid-state sensing membrane on a substrate by means of a semiconductor deposition technology, and polymerizing and fixing polypyrrole on the conductive solid-state membrane by means of an electrochemical polymerization technology. The process according to the invention comprises following steps:
step 1: providing various substrates such as, for example, a insulating material substrate a conductive plate, and selecting an appropriate substrate based mainly on the solid-state sensing material and the sensing environment 1;
step 2: cleaning said substrate 2;
step 3: depositing a solid-state sensing material on the substrate (e.g.: tin dioxide sensing material etc.) 3;
step 4: routing the device 4;
step 5: sealing the material with epoxy resin and fixing the area of a sensing window 5;
step 6: then immersing the device into a electro-polymerization solution, and electro-polymerizing polypyrrole, and thus accomplishing the fabrication of the whole solid-state pH sensing device 6.

In the above-described step (6) for polymerizing polypyrrole, the detail steps are described as follows:
step A: preparing a finished conductive substrate (e.g.: tin dioxide/indium tin oxide glass), wherein the conductivity of surface conductive material 61 is the major consideration for selecting a substrate;
step B: cleaning the substrate 62;
step C: preparing a electro-polymerizing solution, containing a buffer solution, electrolytes, monomer of the conductive polymer (e.g.: phosphate solution, potassium chloride, pyrrole) 63;
step D: connecting the substrate to the positive electrode of a power supply, connecting the platinum electrode to the negative electrode of the power supply, and immerging the substrate into the electro-polymerizing solution while the power supply provides a constant potential which is higher than the oxidizing potential of the conductive polymer (e.g. 4V for electro-polymerizing polypyrrole) for 15 minutes, thus polymerizing the conductive polymer on the substrate 64;
step E: Immerging the polypyrrole sensor into deionized water for 10 minutes to clean the polypyrrole sensor 65;
step F: removing and drying the sensing device, thus completing the fabrication of the polypyrrole sensor 66.

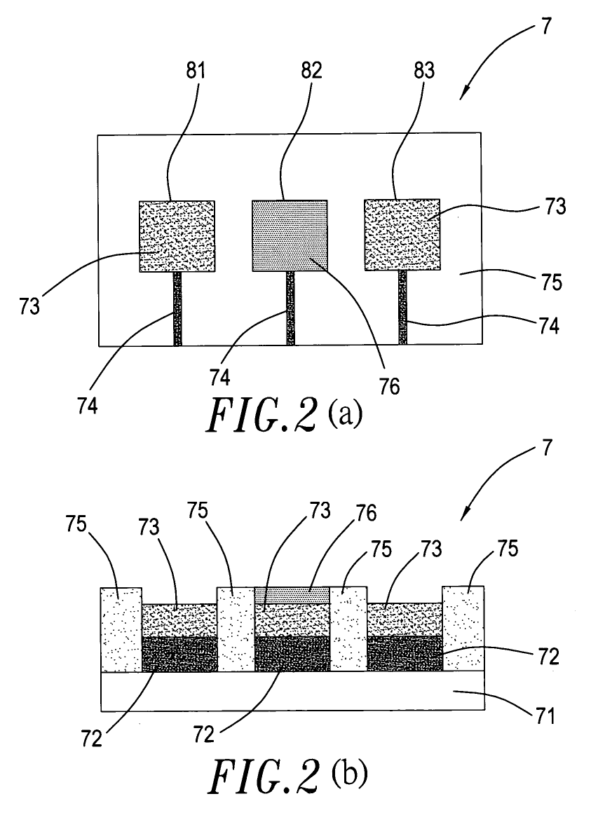

Referring to FIG. 2(a) and FIG. 2(b), there show the top view and sectional view of a whole solid-state pH sensing device fabricated by using polypyrrole as the contrast pH detector, respectively. From the views it can be known that the whole solid-state pH sensing device 7 according to the invention has a tin dioxide sensing membrane 73 deposited on the indium tin oxide 72 of a glass substrate 71, which forms a solid-state ion-sensing electrode for detecting the pH value of a solution, and uses a conductive wire 74 as the signal transmission line, a sealing material, such as epoxy resin 75 and the like, to seal and cover the non-sensing area, and uses encapsulation technology to define the sensing area of the sensing device so as to make a pH sensor and a reference electrode; and thereafter, immerges the finished device into a electro-polymerizing solution of polypyrrole to polymerize the polypyrrole 76 on the tin dioxide sensing membrane 73 and thus completes the fabrication of the polypyrrole pH sensing electrode. The three sensing windows 81, 82, 83 shown in the FIG. 2(a). represent three different electrodes, respectively. Among them, one is reference electrode which uses one tin dioxide sensing window therein for providing the standard reference potential of the sensing device; the other tin dioxide sensing window is used as the pH sensor and has its high sensitivity used for the primary pH sensor. The polypyrrole sensor has a feature that its pH sensing is controllable, which, according to the invention, controls its sensitivity into a steady low sensitivity. By using the features of these three electrodes, the whole solid-state pH electrochemical pH sensing device 7 of the invention can be then constructed.

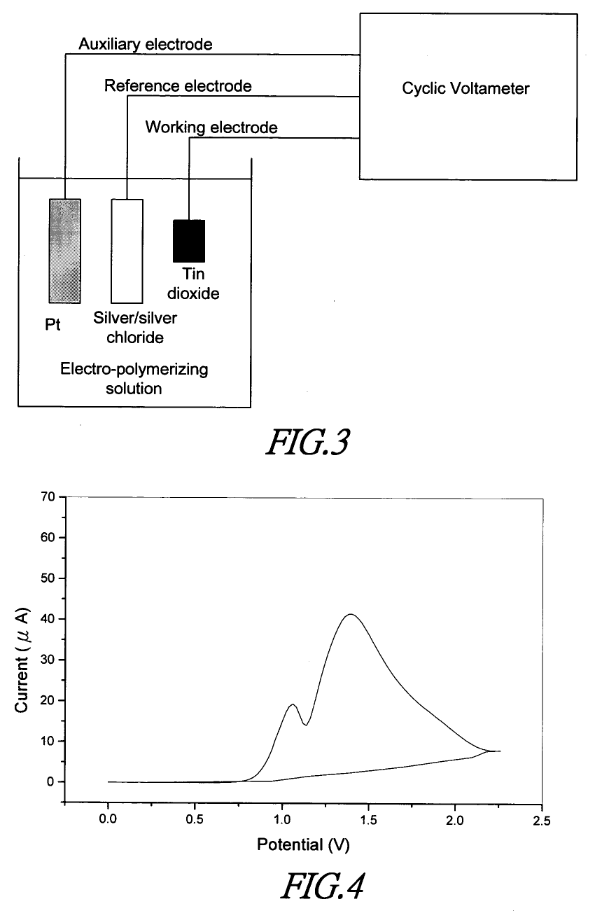

Referring to FIG. 3, a diagram shows the measurement of electro-polymerizing potential of the polypyrrole. From the diagram it can be seen that, by immerging the device into the electro-polymerizing buffer solution that comprises a buffer solution, salts, polypyrrole, etc., under the stable polymerization environment provided by the buffer solution, e.g., phosphate solution, conjugate acid-base solution and the like, and using salts to adjust the conductive feature of the electro-polymerizing solution, e.g.: potassium chloride, sodium chloride, etc, the conductive polymer such as polypyrrole, polyaniline, can be polymerize in the electro-polymerizing solution, and thus fabricates a polypyrrole sensor. Since the pH sensitivity of polypyrrole varies with the electro-polymerizing environment, the sensitivity of polypyrrole can be controlled by adjusting the ratio of electro-polymerizing solution, and a stable differential pair framework pH sensor can thus be fabricated.

Referring to FIG. 4, a diagram shows the measurement system of the oxidizing potential of the polypyrrole. From the diagram it can be known, in order to know whether the electro-polymerizing environment of polypyrrole is suitable, and to select the optimal electro-polymerizing potential, a cyclic voltmeter is used to measure the oxidizing potential of polypyrrole. In the measuring framework diagram, the auxiliary electrode is a platinum electrode, the working electrode is a tin dioxide membrane, and the reference electrode is a silver/silver chloride electrode.

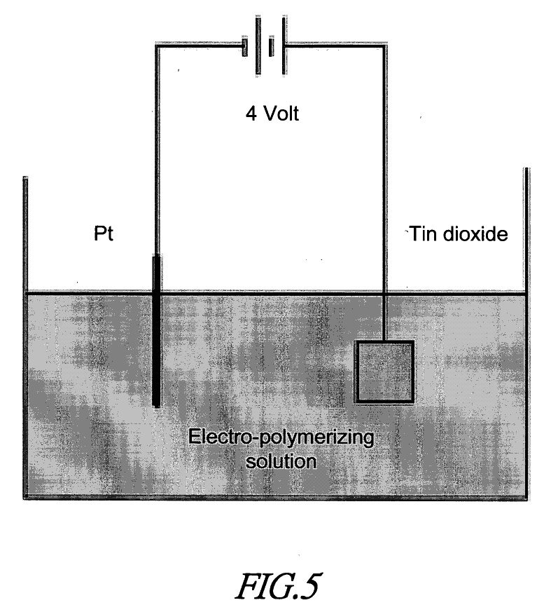

Referring to FIG. 5, a framework diagram shows the electro-polymerization of the whole solid-state pH sensing device. From the diagram it can be known, the characteristic curve is a diagram of the current vs. the potential of polypyrrole. According to the diagram, it can be judged that the oxidizing potential of the polypyrrole is about 1.4 volt. The polypyrrole is super-oxidized if the electro-polymerizing potential is higher than 1.4 volt, which will cause increase of the resistance. Therefore, the invention uses higher potential of 4 volt to electro-polymerize the membrane of the polypyrrole and fabricate a whole solid-state pH Sensing device with lower sensitivity.

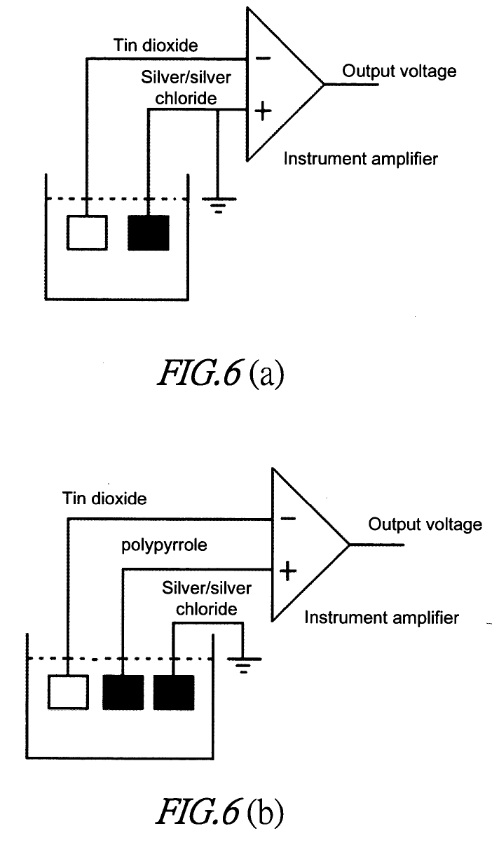

Referring to FIG. 6(a) and FIG. 6(b), there are the characteristic measuring framework diagram of the pH Sensing device and the differential pair framework sensing device, respectively. From the diagrams it can be known that the single sensing device, the tin dioxide sensing device, and the polypyrrole sensor all can get signals from the read-out circuit shown in FIG. 6(a). The read-out circuit uses a circuit with high input impedance, e.g.: MOSFET, operational amplifier, instrumental amplifier, and the like to sense the variation of the surface potential of the sensing device with the pH value of the solution sensed, so that the single sensitivity of the sensing device is obtained. From the complete read-out circuit framework of the whole solid-state pH Sensing device shown in FIG. 6(b), there is a pair of tin dioxide sensing devices in the whole solid-state pH sensing device, wherein one connects to ground, and another connects to the negative input terminal of a instrumental amplifier, and form a reference potential electrode and a pH sensing electrode. Whereas the polypyrrole electrode connects to the positive input terminal of the instrument amplifier, so as to, form the measuring framework of the whole solid-state pH sensing device.

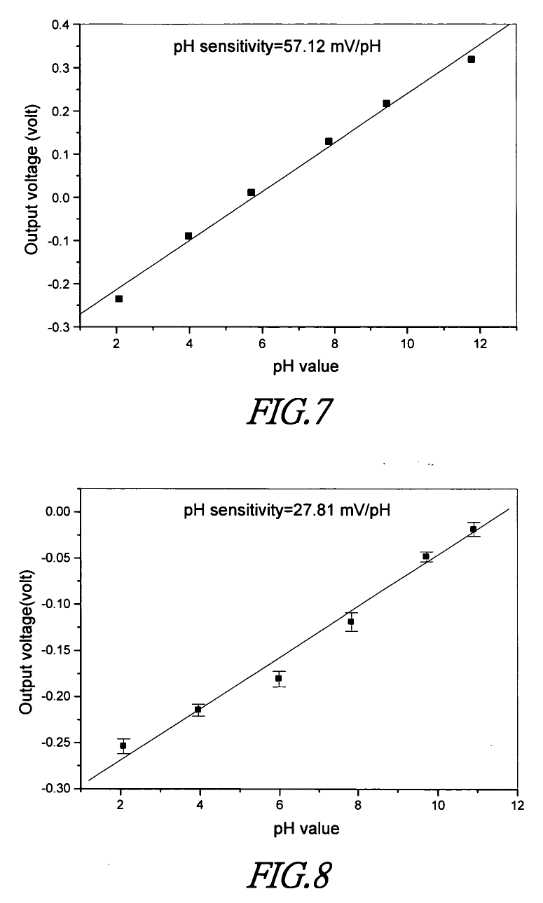

Referring to FIG. 7, a diagram shows the sensitivity calibration curve of the tin dioxide/indium tin oxide glass sensing device. From the diagram it can be known that the characteristic curve is a single sensitivity calibration curve of the tin dioxide/indium tin oxide glass sensing device. According to the graph, it is found that the sensing device has a stable sensitivity and a high sensitivity of 57.1 mV/pH, so that it is suitable for using as the main pH sensing device.

Referring to FIG. 8, a diagram shows the sensitivity curve of the polypyrrole/tin dioxide/indium tin oxide glass sensing device. From the diagram it can be known that the characteristic curve is a sensitivity curve of the polypyrrole/tin dioxide/indium tin oxide glass sensing device. According to the diagram, it is found that the sensing device has stable sensitivity and a low sensing sensitivity of 27.81 mV/pH so that it is suitable for using as the pH sensing device to compare with the whole solid-state pH sensing device.

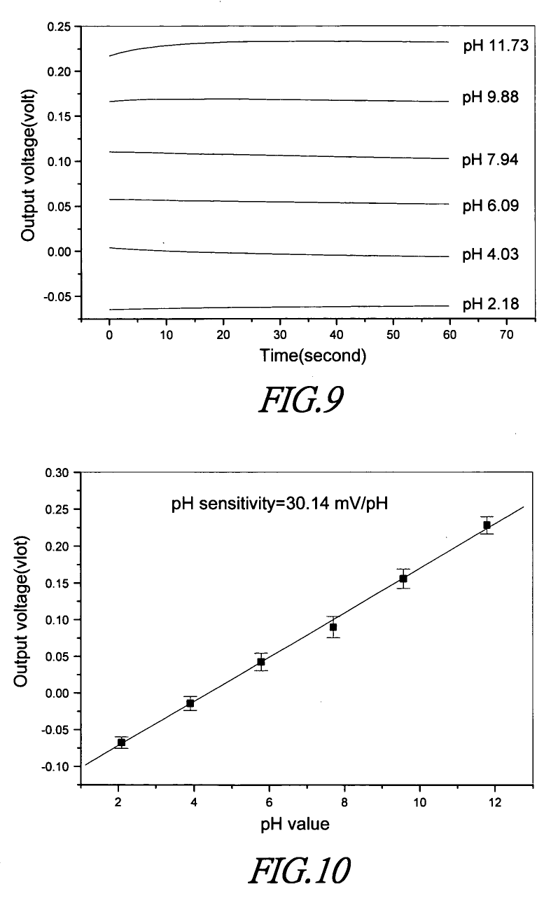

Referring to FIG. 9, a diagram shows sensitivity curves of a whole solid-state pH sensing device fabricated by using polypyrrole as the contrast pH detector. From the diagram it can be known that these characteristic curves are the output potential variation curves of the sensing device in 1 minute when the whole solid-state pH Sensing device immerges into various pH solutions. According to the diagram, it is found that the sensing device has a good stability and the output potential of the sensing device also varies with the pH value of the solution. Accordingly, the sensing device is a good pH sensing device that is suitable for sensing the pH value of the solution to be sensed.

Referring to FIG. 10, a diagram shows the sensitivity curve of a whole solid-state pH sensing device fabricated by using polypyrrole as the contrast pH detector. From the graph it can be known, in order to investigate the stability of the process for fabricating the sensing device, the whole solid-state pH sensing devices thus fabricated is used to measure their sensitivities, respectively. From the diagram, it is found that the sensing device has a good sensing linearity, and each sensing device has small feature error, so that it is a good pH sensing device.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

Reference:

[1] S. Zhang, G. Wright, Y. Yang, “Materials and techniques for electrochemical biosensor design and construction”, Biosensors and Bioelectronics 15,2000, pp.273-282.
[2] Clark L. C., C. Lyois, “Electrode system for continuous monitoring in cardiovascular surgery”, Annals of the New York Academy of Sciences 102, 1962, pp.29-33.
[3] Updike S. J., Hick G. P., “The enzyme electrode”, Nature 214, 1967, pp.986-988.
[4] Manuela Adami, Dario Alliata, Corrado Del Carlo, Mauro Martini, Luciana Piras, Marco Sartore, Claudio Nicolini, “Characterization of silicon transducers with Si₃N₄ sensing surfaces by an AFM and a PAB system”, Sensors and Actuators B, Vol.24-25, 1995, pp.889-893.
[5] A. S. Poghossian, “The super-nemstian pH sensitivity of Ta₂O₅-gate ISFETs”, Sensors and Actuators B, Vol.7, 1992, pp.367-370.
[6] T. Katsube, I. Lauks, J. N. Zemel, “pH-sensitive sputtered iridium oxide films”, Sensors and Actuators B, Vol.2, 1982, pp.399-410.
[7] S. D. Collins, “Practical limits for solid-state reference electrodes”, Sensors and Actuators B, Vol.10, 1993, pp.169-178.
[8] Yuri G. Vlasov, Andrey V. Bratov, “Analytical applications of pH-ISFETs”, Sensors and Actuators B, Vol.10, 1992, pp.1-6.
[9] C. Diekmann, C. Dumschat, K. Cammann, M. Knoll, “Disposable reference electrode”, Sensors and Actuators B, Vol.24-25, 1995, pp.276-278.
[10] C. Cane, A. Gotz, A. Merlos, I. Gracia, A. Errachid, P. Losantos and E. Lora-Tamayo, “Multilayer ISFET membranes for microsystems applications”, Sensors and Actuators B, Vol.35-36, 1996, pp.136-140.
[11] Pavel Neuzil, “ISFET integrated sensor technology”, Sensors and Actuators B, Vol.24-25, 1995, pp.232-235.
[12] Wang Zheng-Xiao, “Applications of penicillinase FET in penicillin-fermentation engineering”, Sensors and Actuators B, Vol.13-14, 1993, pp.568-569.
[13] Andre Haemmerli, Jiri Janata and H. Mack Brown, “Electrical characteristics of K⁺ and Cl⁻ EFT microelectrodes”, Sensors and Actuators, Vol.3, 1982, pp.149-158.
[14] B. H. Van Der Schoot, H. H. Van Den Vlekkert, N. F. De Rooij, A Van Den Berg and A. Grisel, “A flow injection analysis system with glass-bonded ISFETs for the simultaneous detection of calcium and potassium ion and pH”, Sensors and Actuators B, Vol.4, 1991, pp.239-241.
[15] D. Wilhelm, H. Voigt, W. Treichel, R. Ferretti and S. Prasad, “pH sensor based on differential measurements on one pH-FET chip”, Sensors and Actuators B, Vol.4, 1991, pp.145-149.
[16] V. Tvarozek, H. Ti Tien, I. Novotny, T. Hianik, J. Dlugopolsky, W. Ziegler, A. Leitmannnova-Ottova, J. Jakabovic, V. Rehacek and M. Uhlar, “Thin-film microsystem applicable in (bio)chemical sensors”, Sensors and Actuators B, Vol.18-19, 1994, pp.597-602.
[17] Werner Moritz, Fred Lisdat, Bart H. Van Der Schoot, Nico F. De Rooij, Hans H. Van Den Vlekkert, H.C.G. Ligtenberg, Ingolf Grohmann, “Flow injection analysis using pH/pF ISFET combinations for determination of very low fluoride concentrations”, Sensors and Actuators B, Vol.15-16, 1993, pp.223-227.
[18] J.Van Der Spiegel, I. Lauks, P. Chan, and D. Babic, “The extended gate chemical sensitive field effect transistor as multi-species microprobe”, Sensors and Actuators, Vol.4, 1983, pp.291-298.
[19] Huixian Zhu, Tai-Chin Lo, Ralf Lenigk, Reinhard Renneberg, “Fabrication of a novel oxygen sensor with CMOS compatible processes”, Sensors and Actuators B, Vol. 46, 1998, pp. 155-159.
[20] Joseph J. Pancrazio, Paul P. Bey Jr, Arash Loloee, Subba Rao Manne, Hui-Chuan Chao, Lorn L. Howard, “Description and demonstration of a CMOS amplifier-based-system with measurement and stimulation capability for bioelectrical signal transduction”, Biosensors and Bioelectronics, Vol. 13, 1998, pp. 971-979.
[21] N. Zine, A. Ivorra, J. Aguilo, R. Villa, J. Millan, J. Bausells, A. Errachid, P. Godignon, A. Benvenuto, L. Beccai, F. Valvo, A. Menciassi, P. Dario, M. C. Carrozza, “Multisensor silicon needle for cardiac applications”, 1st Annual International Conference On Microtechnologies in Medicine and Biology, 2000, pp. 216-219.
[22] R. J. Reay, S. P. Kounaves, G. T. A. Kovacs, “An integrated CMOS potentiostat for miniaturized electroanalytical instrumentation”, IEEE International Solid-State Circuits Conference, 1994, pp. 162-163.

1. A process for fabricating a whole solid-state pH sensing device by using polypyrrole as the contrast pH detector, said process comprising following steps:
step 1: preparing various solid-state substrates and selecting an appropriate substrate based on the solid-state sensing material and the sensing environment;
step 2: depositing a solid-state sensing material on said substrate;
step 3: routing the device;
step 4: using a epoxy resin to seal the material and fixing the sensing window area; and
step 5: then immersing the device into a electro polymerizing solution, and electro-polymerizing polypyrrole, thus completing the fabrication of the whole solid-state pH sensing device.

2. A process for fabricating a whole solid-state pH sensing device by using the polypyrrole as the contrast pH detector as recited in claim 1, wherein the step of electro-polymerizing polypyrrole comprises following steps:
step A: preparing said finished conductive substrate;
step B: preparing said electro-polymerizing solution, which comprises a buffer solution, electrolytes, the monomer of polypyrrole;
step D: connecting the substrate to the positive electrode of the power supply, and connecting a platinum electrode to the negative electrode of the power supply, and immerging the substrate into said electro-polymerizing solution, where the power supply provides a constant potential which is higher than the oxidizing potential of said polypyrrole, in a manner that said polypyrrole can be polymerized on said substrate;
step E: immerging the polypyrrole sensor into the de-ionized water to clean said polypyrrole sensor;
step F: removing and drying said sensing device, thus completing the fabrication of the polypyrrole sensor.

3. A process for fabricating a whole solid-state pH sensing device by using the polypyrrole as the contrast pH detector as recited in claim 1, wherein said solid-state substrate is selected from the group consisting of a silicon substrate, a glass substrate, a ceramic substrate or a plastic substrate.

4. A process for fabricating a whole solid-state pH sensing device by using the polypyrrole as the contrast pH detector as recited in claim 1, wherein said sensing material is selected from the group consisting of a tin dioxide membrane or other solid-state conductive ion-sensing membrane.

5. A process for fabricating a whole solid-state pH sensing device by using the polypyrrole as the contrast pH detector as recited in claim 1, wherein said polymerizing solution of the polypyrrole comprises a buffer solution, salts, polypyrrole, such as the electro-polymerizing solution comprising a phosphate solution, potassium chloride, and polypyrrole; wherein, through changing the composition of said polymerizing solution, the control of the sensitivity of said polypyrrole sensor can be achieved, and wherein this technology can be applied to fabricate the corresponding sensing electrode with an appropriate sensitivity and the control of the sensitivity of the differential pair pH sensing device can be obtained.

6. A process for fabricating a whole solid-state pH sensing device by using the polypyrrole as the contrast pH detector, said process comprises:
depositing a non-insulating solid-state ion-sensing membrane on a insulating substrate or non-insulating substrate;
using a conductive wire as the signal transmission line;
using a seal material such as a epoxy resin to seal and coat the non-sensing area;
using encapsulation technology to define the sensing area of the sensing device to fabricate the pH sensor and the reference electrode;
thereafter, immerging the finished device into a polymerizing solution of polypyrrole, and polymerizing polypyrrole on a tin dioxide membrane, thus complete the fabrication of the polypyrrole sensor;
wherein by virtue of the electrode feature formed from three sensing windows, said differential pair electrochemical pH Sensing device is thus constructed.

7. A process for fabricating a whole solid-state pH sensing device by using the polypyrrole as the contrast pH detector, said process as recited in claim 6, wherein said three sensing windows are a reference electrode, a polypyrrole sensor and a pH sensor.

8. A process for fabricating a whole solid-state pH sensing device by using the polypyrrole as the contrast pH detector, said process as recited in claim 6, wherein said electrodes are all solid-state electrodes, and are planar frameworks, do not need to immerge in the buffer solution for storage, and hence is easy to preserve and the feature is unlikely affected by the environmental interference.
